(19) United States
(12) Patent Application Publication
(10) Pub. No.: US 2020/0395129 A1
(43) Pub. Date: Dec. 17, 2020

(54) SYSTEMS AND METHODS FOR IDENTIFICATION OF CLINICALLY SIMILAR INDIVIDUALS, AND INTERPRETATIONS TO A TARGET INDIVIDUAL

(71) Applicant: Medial Research Ltd., Kfar-Malal (IL)

(72) Inventors: Nir KALKSTEIN, Herzlia (IL); Avi SHOSHAN, Mazkeret Batia (IL); Udi BOBROVSKY, SaAd (IL)

(73) Assignee: Medial Research Ltd., Kfar-Malal (IL)

(21) Appl. No.: 16/971,723

(22) PCT Filed: Aug. 13, 2018
(86) PCT No.: PCT/IL2018/050898

§ 371 Date: Aug. 21, 2020

Related U.S. Application Data
(60) Provisional application No. 62/545,499, filed on Aug. 15, 2017.

(51) Int. Cl. G16H 50/70; G16H 50/30; G16H 10/60; G16H 50/20; G06K 9/62

(52) U.S. Cl. CPC G16H 50/70; G16H 50/30; G06K 9/6276; G16H 50/20; G06K 9/6215; G16H 10/60

(57) ABSTRACT

There is provided a method, comprising: receiving an indication of medical test results of a target individual, applying a trained classifier to the indication to compute a set of clinical outcome prediction scores, wherein each respective score is indicative of a prediction of a certain pathology of a certain physiological system of the target individual, computing for the set of clinical outcome prediction scores of the target individual, a cluster of sets of computed clinical output predictions for individuals, according to a requirement of a statistical distance, wherein each set of clinical outcome prediction scores is indicative of a predicted co-morbidity of pathologies of physiological systems, wherein the individuals defined by the computed cluster denote individuals clinically similar to the target individual in terms of predicted co-morbidity of the plurality of pathologies of the plurality of physiological systems, and computing an aggregation of medical parameter(s) of the cluster.

### FIELD AND BACKGROUND OF THE INVENTION

The present invention, in some embodiments thereof, relates to analysis of medical test results and, more specifically, but not exclusively, to systems and methods for aggregation of a medical parameter of a cluster of individuals clinically similar to a target individual.

Traditionally, medical test results of an individual are interpreted in isolation, for the individual alone. The medical tests results are examined to find values that are normal or that deviate from normal values. The interpreted normal or abnormal values may suggest a previously un-thought of diagnosis, may confirm a hypothesis of a diagnosis, may refute a diagnosis, and/or may warrant further investigation (e.g., ordering additional tests).

### SUMMARY OF THE INVENTION

According to a first aspect, a method of providing a client terminal with an aggregation of at least one medical parameter of members of a cluster of individuals clinically similar to a target individual, comprises: receiving by a computing system in communication with a dataset storing a set of computed clinical outcome prediction scores for each of a plurality of individuals, via a network, an indication of medical test results of the target individual, applying a trained classifier to the indication of medical test results to compute, for the target individual, a set of clinical outcome prediction scores, wherein each respective score is indicative of a prediction of a certain pathology of a plurality of pathologies of a certain physiological system of a plurality of physiological systems of the target individual, computing for the set of clinical outcome prediction scores of the target individual, a cluster of sets of computed clinical output predictions stored by the dataset, wherein the cluster of sets of computed clinical outcome prediction scores is computed according to a requirement of a statistical distance of sets of computed clinical outcome prediction scores stored by the dataset relative to the set of clinical outcome prediction scores of the target individual, wherein each set of clinical outcome prediction scores is indicative of a predicted co-morbidity of a plurality of pathologies of a plurality of physiological systems, wherein the individuals defined by the computed cluster denote individuals clinically similar to the target individual in terms of predicted co-morbidity of the plurality of pathologies of the plurality of physiological systems, computing an aggregation of at least one medical parameter of the cluster, and outputting the aggregation for presentation by the client terminal.

According to a second aspect, a system for providing a client terminal with an aggregation of at least one medical parameter of members of a cluster of individuals clinically similar to a target individual, comprises: a non-transitory memory having stored thereon a code for execution by at least one hardware processor of a computing system in communication with a dataset storing a set of computed clinical outcome prediction scores for each of a plurality of individuals, the code comprising: code for receiving an indication of medical test results of the target individual, code for applying a trained classifier to the indication of medical test results to compute, for the target individual, a set of clinical outcome prediction scores wherein each respective score is indicative of a prediction of a certain pathology of a plurality of pathologies of a certain physiological system of a plurality of physiological systems of the target individual, computing for the set of clinical outcome prediction scores of the target individual, a cluster of sets of computed clinical output predictions stored by the dataset, wherein the cluster of sets of computed clinical outcome predictions is computed according to a requirement of a statistical distance of sets of computed clinical outcome prediction scores stored by the dataset relative to the set of clinical outcome prediction scores of the target individual, wherein each set of clinical outcome prediction scores is indicative of a predicted co-morbidity of the plurality of pathologies of the plurality of physiological systems, wherein the individuals defined by the computed cluster denote individuals clinically similar to the target individual in terms of predicted co-morbidity of the plurality of pathologies of the plurality of physiological systems, and computing an aggregation of at least one medical parameter of the cluster, and code for outputting the aggregation for presentation by the client terminal.

According to a third aspect, a computer program product for providing a client terminal with an aggregation of at least one medical parameter of members of a cluster of individuals clinically similar to a target individual, comprises: a non-transitory memory having stored thereon a code for execution by at least one hardware processor of a computing system in communication with a dataset storing a set of computed clinical outcome prediction scores for each of a plurality of individuals, the code comprising: instructions for receiving an indication of medical test results of the target individual, instructions for applying a trained classifier to the indication of medical test results to compute, for the target individual, a set of clinical outcome prediction scores wherein each respective score is indicative of a prediction of a certain pathology of a plurality of pathologies of a certain physiological system of a plurality of physiological systems of the target individual, computing for the set of clinical outcome prediction scores of the target individual, a cluster of sets of computed clinical output predictions stored by the dataset, wherein the cluster of sets of computed clinical outcome predictions is computed according to a requirement of a statistical distance of sets of computed clinical outcome prediction scores stored by the dataset relative to the set of clinical outcome prediction scores of the target individual, wherein each set of clinical outcome prediction scores is indicative of a predicted co-morbidity of the plurality of pathologies of the plurality of physiological systems, wherein the individuals defined by the computed cluster denote individuals clinically similar to the target individual in terms of predicted co-morbidity of the plurality of pathologies of the plurality of physiological systems, and computing an aggregation of at least one medical parameter of the cluster, and instructions for outputting the aggregation for presentation by the client terminal.

The systems and/or methods and/or code instructions described herein provide a technical solution to the technical problem of analyzing medical test results and/or other data of individuals stored in a medical database, which may include a large number of records, for example, over a million individuals, over 10 million individuals, or other numbers.

The records of the larger number of individuals are analyzed to extract clinically relevant data and/or healthcare insights for a target individual, for example, to predict clinical outcomes that the target individual is at increased risk for, to determine an overall picture of the state of the patient, to determine additional medical interventions (e.g., specialist consolations, prescription medications), and/or to improve analysis of measured analytes.

Using standard methods, analysis of the large number of records of individuals may be inaccurate for determining clinical outcomes that the target individual is at increased risk for, an overall picture of the state of the patient, and/or to additional medical interventions (e.g., specialist consolations, prescription medications). For example, the large number of individuals may include individuals that have a health status that is different than the target individuals. Inclusion of results from such individuals may skew the results towards an inaccurate conclusion.

The systems and/or methods and/or code instructions described herein improve computer-related technology, namely, improve performance of a computing device analyzing a medical database storing large number of individual records and/or storing a large amount of patient medical data, (e.g., at least 1 million, or at least 10 million, or other number of records of individuals).

The systems and/or methods and/or code instructions described herein select a cluster of a relatively smaller number of individuals (e.g., 1000) for analysis, rather than analyzing the larger set of stored records (e.g., 1 million, 10 million).

Analysis of the cluster is more computationally efficient than analysis of the larger set of records. For example, rather than computing a prediction for each query (e.g., question for which an answer is sought), once the cluster is computed, indications represented by the different between the cluster and the population may be measured. Improved computational performance is obtained, for example, in terms of decreased processing time and/or decreased processor utilization in analyzing the cluster in comparison to analysis of the larger number of records.

The computational efficiency is improved, for example, when multiple aggregations and/or analyses of the cluster are performed in comparison to performing the aggregation and/or analyses on the larger set. For example, computation of the biological age and/or physiological system age, computation of medical interventions (e.g., specialist consolations, prescription medications), and/or aggregation of values of measured analytes.

Moreover, analysis of the cluster is more accurate than analysis of the larger set of records, since the computed cluster includes clinical outcome prediction scores of individuals that are clinically more similar to the target individual that the remaining individuals. Inclusion of clinical outcome prediction scores for the remaining individuals reduces the accuracy of the analysis. For example, for an elderly sick individual with multiple co-morbidities, inclusion of data from young health individuals skews the analysis.

In a further implementation form of the first, second, and third aspects, the dataset of clinical outcome prediction scores of the plurality of individuals are arranged as an n-dimensional Euclidean space, wherein each of the n dimensions denotes an axis according to a respective clinical outcome prediction indicative of a certain pathology of each certain physiological system, wherein each individual is represented as a point in the n-dimensional Euclidean space according to respective prediction scores of each axis, wherein the computing the cluster is performed by mapping the set of clinical outcome prediction scores for the target individual to a point in the n-dimensional Euclidean space and computing the nearest neighbors based on closest Euclidean distance between the point denoting each individual and the point denoting the target individual.

In a further implementation form of the first, second, and third aspects, the requirement of the statistical distance defines a predefined number of nearest neighbors according to Euclidean distances between a point in the n-dimensional space denoting the mapped set of clinical outcome prediction scores for the target individuals and points in the n-dimensional space noting respective locations of each of the members of the cluster.

In a further implementation form of the first, second, and third aspects, the cluster is performed by at least one processor executing code instructions based on a k-nearest neighbor (KNN) method.

In a further implementation form of the first, second, and third aspects, the clinical outcome prediction scores of the target individual are indicative of the probability of the target individual developing the corresponding predicted certain pathology of the certain physiological system within a predefined future time interval.

In a further implementation form of the first, second, and third aspects, the method further comprises and/or the system further includes code for and/or the computer program product includes additional instructions for excluding from the plurality of individuals, individuals having indications of medical test results separated from a defined clinical outcome stored in a medical database by greater than a predefined interval of time.

In a further implementation form of the first, second, and third aspects, the trained classifier comprises a gradient boosting classifier.

In a further implementation form of the first, second, and third aspects, the method further comprises and/or the system further includes code for and/or the computer program product includes additional instructions for computing the trained classifier by: extracting from a medical database, for a subset of the plurality of individuals, at least one set of indications of medical test results and at least one indication of a diagnosis of a clinical outcome, wherein individuals without the diagnosis of the clinical outcome are labeled as negative individuals denoting lack of association with the clinical outcome and individuals associated with the diagnosis are labeled as positive individuals denoting an association with the clinical outcome, creating a training dataset by sampling a defined ratio of individuals labeled as positive individuals and individuals labeled as negative individuals, and computing the trained classifier according to the training dataset.

In a further implementation form of the first, second, and third aspects, the method further comprises and/or the system further includes code for and/or the computer program product includes additional instructions for individuals labeled as positive individuals filtering out members of the set of indications of medical test results with dates that are outside of a defined time interval relative to the date of the diagnosis of clinical outcome, and for individuals labeled as negative individuals filtering out members of the set of indications of medical test results with dates that are within the defined time interval relative to the date of computation of the trained classifier.

In a further implementation form of the first, second, and third aspects, the method further comprises and/or the system further includes code for and/or the computer program product includes additional instructions for accessing from a medical database, for each of the subset of the plurality of individuals, at least one of: at least one demographic parameter and at least one prescribed medication, and including the at least one of: the at least one demographic parameter and the at least one prescribed medication in the training dataset.

In a further implementation form of the first, second, and third aspects, the method further comprises and/or the system further includes code for and/or the computer program product includes additional instructions for computing the dataset storing the set of clinical outcome prediction scores, by applying the trained classifier to compute the clinical outcome prediction scores for each member of a validation dataset including individuals of the medical database excluded from the training dataset.

In a further implementation form of the first, second, and third aspects, the method further comprises and/or the system further includes code for and/or the computer program product includes additional instructions for designating a calibration set of a plurality of individuals associated with medical test results stored in a medical database, applying the trained classifier to the calibration set to compute a first set of clinical outcome prediction scores, applying a demographic classifier to demographic data of the calibration set to compute a second set of clinical outcome prediction scores, wherein the demographic classifier computes clinical outcome prediction scores according to demographic data of a certain individual, sorting the first and second set of computed clinical outcome prediction scores computed for the calibration set, dividing the sorted clinical outcome prediction scores into bins, computing the prevalence of each clinical outcome prediction indicative of a certain pathology of each certain physiological system in the calibration set, and calibrating each bin according to the computed prevalence of clinical prediction outcomes of the respective bins.

In a further implementation form of the first, second, and third aspects, the method further comprises and/or the system further includes code for and/or the computer program product includes additional instructions for computing a Fisher statistical significance of prevalence of clinical outcome predictions indicative of a certain pathology of each certain physiological system of the cluster relative to clinical outcome predictions of a demographic classifier applied to the demographic data of the target individual, wherein the demographic classifier computes clinical outcome predictions according to demographic data of a certain individual, and excluding clinical outcome predictions with Fisher p-values above a threshold, wherein the remaining clinical outcome predictions denote an elevated risk of the target individual developing the remaining clinical outcome predictions.

In a further implementation form of the first, second, and third aspects, the method further comprises and/or the system further includes code for and/or the computer program product includes additional instructions for computing a ratio of the prevalence of clinical outcome predictions indicative of a certain pathology of each certain physiological system of the cluster relative to clinical outcome prediction scores computed by applying a demographic classifier to the demographic data of the target individual, wherein the demographic classifier computes clinical outcome prediction scores according to demographic data of a certain individual, and excluding clinical outcome prediction scores having a computed ratio below a predefined value, wherein the remaining clinical outcome prediction scores denote an elevated risk of the target individual developing the remaining clinical outcome predictions.

In a further implementation form of the first, second, and third aspects, the method further comprises and/or the system further includes code for and/or the computer program product includes additional instructions for identifying clinical outcome predictions indicative of a certain pathology of each certain physiological system with a prediction score value above a requirement, wherein the identified clinical outcome prediction scores denote an absolute risk of the target individual developing the respective clinical outcome prediction.

In a further implementation form of the first, second, and third aspects, the certain pathology of each certain physiological system include one or more members selected from the group consisting of: death, neoplasm, ischemic heart disease, type two diabetes, liver disease, chronic renal failure, chronic obstructive pulmonary disease, acquired hypothyroidism, epilepsy, migraine, chronic fatigue syndrome, trigeminal neuralgia, hypertensive disease, nervous system disease, acute sinusitis, bell facial palsy, carpal tunnel syndrome, retinal detachment, diabetic retinopathy, degeneration of macula, glaucoma, vertiginous syndromes, hyperthyroidism, acute renal failure, heart valve disorders, haematuria, psoriasis, systemic lupus erythematosus, polymyalgia rheumatic, pulmonary embolism, cataract, cardiac dysrhythmias, and osteoporosis.

In a further implementation form of the first, second, and third aspects, computing the aggregation of at least one medical parameter comprises computing a prevalence for at least one clinical outcome prediction indicative of a certain pathology of each certain physiological system for the cluster of clinically similar individuals.

In a further implementation form of the first, second, and third aspects, computing the aggregation of the at least one medical parameter comprises computing a difference between a prevalence of at least one clinical outcome prediction computed for the cluster in comparison to the prevalence of the at least one clinical outcome prediction computed for a general population that is demographically correlated with the target individual, and generating an indication of the at least one clinical outcome prediction when the prevalence of the at least one clinical outcome prediction is statistically significant between the cluster and the general population.

In a further implementation form of the first, second, and third aspects, computing the aggregation comprises computing an average age of members of the cluster of clinically similar individuals, wherein the average age of members of the cluster of clinically similar individuals denotes a biological age of the target individual.

In a further implementation form of the first, second, and third aspects, computing the aggregation comprises computing an average age of members of the cluster of clinically similar individuals that are correlated according to a requirement with at least one clinical prediction indicative of a certain pathology of each certain physiological system computed for the target individual, wherein the computed average age denotes a biological age of at least one of an organ and a physiological system of the target individual associated with each respective clinical prediction.

In a further implementation form of the first, second, and third aspects, computing the aggregation of the at least one medical parameter comprises for at least one of an indication of at least one of a medical treatment, a prescribed medication, and a specialist consultation of the target individual, computing a difference between an incidence of at least one of the medical treatment, the prescribed medication, and the specialist consultation computed for the cluster in comparison to an incidence of at least one of the medical treatment, the prescribed medication, and the specialist consultation computed for a general population that is demographically correlated with the target individual, and generating an indication of the at least one of the medical treatment, the prescribed medication, and the specialist consultation when the incidence of the at least one of the medical treatment, the prescribed medication, and the specialist consultation is statistically significant between the cluster and the general population.

In a further implementation form of the first, second, and third aspects, computing the aggregation of the at least one medical parameter comprises for at least one of an indication of at least one of a medical treatment, a prescribed medication, and a specialist consultation of the target individual, computing the effect of the contribution of at least one of the medical treatment, the prescribed medication, and the specialist consultation in a difference between a prevalence of at least one clinical outcome prediction computed for the cluster in comparison to the prevalence of the at least one clinical outcome prediction computed for a general population that is demographically correlated with the target individual, and generating an indication of the at least one clinical outcome prediction and the at least one of the medical treatment, the prescribed medication, and the specialist consultation when the effect of the contribution on the prevalence of the at least one clinical outcome prediction is statistically significant between the cluster and the general population.

In a further implementation form of the first, second, and third aspects, computing the aggregation of the at least one medical parameter comprises for at least one of a value and a trend of values of an analyte included in the indication of laboratory test of the medical test results of the target individual, computing the effect of the contribution of at least one of the value and the trend of values of the analyte in a difference between a prevalence of at least one clinical outcome prediction computed for member of the cluster having similar at least one value and trend of the analyte, in comparison to the prevalence of the at least one clinical outcome prediction computed for a general population that is demographically correlated with the target individual, and generating an indication of the at least one of the value and the trend of the analyte and the at least one clinical outcome prediction when the effect of the contribution on the prevalence of the at least one clinical outcome prediction is statistically significant between the cluster and the general population.

In a further implementation form of the first, second, and third aspects, the clinical outcome prediction scores are computed for the target individuals and the plurality of individuals according to the indication of medical test results and indications of determinant of health data including one or more of: demographic data, genetic data, nutrition, and environmental exposure.

In a further implementation form of the first, second, and third aspects, the medical tests are selected from the group consisting of: laboratory tests, physical exam findings, symptoms obtained from a medical history, radiological examination findings, and other medical tests and/or measurements performed by a medical device.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method of providing a client terminal with an aggregation of at least one medical parameter of a cluster of individuals clinically similar to a target individual in response to an indication of current medical test results of the target individual, in accordance with some embodiments of the present invention;

FIG. 2 is a block diagram of components of a system for providing a client terminal with an aggregation of at least one medical parameter of a cluster of individuals clinically similar to a target individual in response to an indication of current medical test results of the target individual, in accordance with some embodiments of the present invention;

FIG. 3 is a schematic of an exemplary GUI presenting computation of the biological age of the target individual based on an aggregation of the age of members of the cluster, in accordance with some embodiments of the present invention;

FIG. 4 is a schematic of an exemplary GUI presenting computation of the prevalence of clinical outcome(s) of the cluster statistically significantly different from clinical outcomes of a demographically correlated subset of the general population, in accordance with some embodiments of the present invention;

FIG. 5 is a schematic of an exemplary GUI presenting computation of the incidence of specialist consultations of the cluster that are statistically significantly different from specialist consultations of a demographically correlated subset of the general population, in accordance with some embodiments of the present invention;

FIG. 6 is a schematic depicting an application running on a smartphone displaying the aggregated value computed from the cluster, for each analyte measured by the medical tests obtained for the target individual, relative to a distribution of the value of the analyte in the general population demographically correlated with the target individual, in accordance with some embodiments of the present invention;

FIG. 7 is a schematic of an exemplary GUI that presents prevalence of clinical outcome predictions in association with a graph depicting trends in values of analytes measured in laboratory tests and/or changes to medical data, and medical history, in accordance with some embodiments of the present invention;

FIG. 8 is a schematic depicting a social network application displayed on a smartphone for clinically similar individuals, in accordance with some embodiments of the present invention; and

FIG. 9 is a schematic depicting a report and/or GUI presenting a summary of multiple aggregations computed based on the identified cluster, according to the medical test results of the target individual, in accordance with some embodiments of the present invention;

FIG. 10 is a schematic depicting applications provided by a server to client terminals of users over a network, based on aggregation of data of a cluster of individuals clinically similar to a target individual according to medical test results of the target individual, in accordance with some embodiments of the present invention;

FIG. 11 is a graph depicting computation of 1000 individuals clinically similar to the target individual, in accordance with some embodiments of the present invention;

FIG. 12A is a graph indicating a region of values for which members of the cluster having lymphocyte values falling therein have a statistically significant difference in comparison to the general, in accordance with some embodiments of the present invention; and

FIG. 12B is an example of a graph for an exemplary computation of the value and/or trend of the analyte for which the prevalence of the clinical outcome prediction(s) is statistically significant between the members of the cluster and the general population, in accordance with some embodiments of the present invention.

### DESCRIPTION OF SPECIFIC EMBODIMENTS OF THE INVENTION

The present invention, in some embodiments thereof, relates to analysis of medical test results and, more specifically, but not exclusively, to systems and methods for aggregation of a medical parameter of a cluster of individuals clinically similar to a target individual.

An aspect of some embodiments of the present invention relates to systems, methods, and/or code instructions stored in a data storage device executable by one or more processors, for computing an aggregation of medical parameter(s) from a cluster of sets of clinical outcome prediction scores of individuals that are clinically similar to a target individual in terms of predicted co-morbidity of multiple pathologies of multiple physiological systems. Each respective score is indicative of a prediction of a certain pathology of multiple pathologies of a certain physiological system of multiple physiological systems of the respective individual.

The cluster is computed according to a requirement of a statistical distance of sets of clinical outcome prediction scores (stored in a dataset) relative to a set of clinical outcome prediction scores computed for the target individual. The clinical outcome prediction scores are each indicative of the respective individual developing the respective clinical outcome prediction (i.e., the certain pathology of the certain physiological system) within a predefined future time interval (e.g., 1, 3, or 5 years). Each set of clinical outcome prediction scores is indicative of a predicted co-morbidity of multiple pathologies of multiple physiological systems.

The set of clinical outcome prediction scores for the target individual is computed by applying a trained classifier to medical test results of the target individual. The medical parameter(s) is computed for the cluster by aggregating data from the cluster. For example, the biological age of the target individual is computed as an average of the age of individual members of the cluster.

The biological age may denote a more accurate picture of the overall state of the target individual. In another example, the prevalence of one or more medical conditions (e.g., defined by a medical diagnosis) of individuals of the cluster may be computed.

The prevalence of the medical condition(s) in individuals of the cluster may denote medical condition(s) for which the target individual is at increased risk. In yet another example, the incidence of medical treatments and/or specialist consultations and/or prescribed medications for individuals of the cluster may be determined.

The incidence of medical treatments and/or specialist consultations and/or prescribed medications may denote recommended medical treatments and/or specialist consultations and/or prescribed medications for the target individual. In yet another example, the value of an analyte measured in the medical tests of the target individual, is computed by aggregating values of corresponding analytes for individuals of the set members of the cluster that are demographically correlated with the target individual, for example within a similar age range and/or similar gender. The aggregated value of the analyte may improve analysis of the target individual's measured value, for example, whether the value is reasonable, normal, abnormal, or a medial computational reference value.

Optionally, the cluster of sets of computed clinical outcome prediction scores denoting individuals clinically similar to the target individual is identified by computing the nearest neighbors to a point representing the target individual, in an n-dimensional Euclidean space. The n-dimensional Euclidean space is a representation of the dataset storing the clinical outcome prediction scores computed for the population of individuals. Each of the n-dimensions denotes an axis of a respective clinical outcome prediction score. Each individual of the population is represented by a point mapped to the n-dimensional space according to the clinical outcome prediction scores of the respective individual.

The point representing the target individual is mapped into the n-dimensional Euclidean space according to the clinical outcome prediction scores computed by the classifier applied to the medical test results. The location of the point relative to each axis of the n-dimensional space may be defined according to the value of the predictive score.

The clinical outcome prediction scores are indicative of the probability of the respective individual developing the respective clinical outcome (i.e., the certain pathology of the certain physiological system) within a predefined future time interval. Each individual is represented as the value of the respective axis denoting the clinical outcome score of the respective dimension of the n-dimensional Euclidean space.

The classifier may be trained on a sub-set of individuals having medical test result records stored in the medical database. The dataset of clinical outcome prediction scores for the population of individuals may be created by applying the trained classifier to another set of records of individuals (excluding the records of individuals used to train the classifier).

Optionally, the medical parameter(s) is presented of a display in comparison to a computed medical parameter(s) for a general population that is optionally demographically correlated with the target individual. Statistically significant differences between the cluster and the general population (optionally demographically correlated with the target individual) may be identified. For example, prevalence of a certain medical condition may be statistically significantly higher (or lower) in the cluster in comparison to the general population.

The cluster is at increased risk or decreased risk of the certain medical condition relative to the general population. Since the cluster represents individuals that are clinically similar to the target individual, the target individual is at increased risk or decreased risk of developing the certain medical condition.

The systems and/or methods and/or code instructions described herein provide a technical solution to the technical problem of analyzing medical test results and/or other data of individuals stored in a medical database, which may include a large number of records, for example, over a million individuals, over 10 million individuals, or other numbers.

The records of the larger number of individuals are analyzed to extract clinically relevant data and/or healthcare insights for a target individual, for example, to predict clinical outcomes that the target individual is at increased risk for, to determine an overall picture of the state of the patient, to determine additional medical interventions (e.g., specialist consolations, prescription medications), and/or to improve analysis of measured analytes.

Using standard methods, analysis of the large number of records of individuals may be inaccurate for determining clinical outcomes that the target individual is at increased risk for, an overall picture of the state of the patient, and/or to additional medical interventions (e.g., specialist consolations, prescription medications). For example, the large number of individuals may include individuals that have a health status that is different than the target individuals. Inclusion of results from such individuals may skew the results towards an inaccurate conclusion.

The systems and/or methods and/or code instructions described herein improve computer-related technology, namely, improve performance of a computing device analyzing a medical database storing large number of individual records and/or storing a large amount of patient medical data, (e.g., at least 1 million, or at least 10 million, or other number of records of individuals).

The systems and/or methods and/or code instructions described herein select a cluster of a relatively smaller number of individuals (e.g., 1000) for analysis, rather than analyzing the larger set of stored records (e.g., 1 million, 10 million). Analysis of the cluster is more computationally efficient than analysis of the larger set of records. For example, rather than computing a prediction for each query (e.g., question for which an answer is sought), once the cluster is computed, indications represented by the different between the cluster and the population may be measured.

Improved computational performance is obtained, for example, in terms of decreased processing time and/or decreased processor utilization in analyzing the cluster in comparison to analysis of the larger number of records.

The computational efficiency is improved, for example, when multiple aggregations and/or analyses of the cluster are performed in comparison to performing the aggregation and/or analyses on the larger set. For example, computation of the biological age and/or physiological system age, computation of medical interventions (e.g., specialist consolations, prescription medications), and/or aggregation of values of measured analytes.

Moreover, analysis of the cluster is more accurate than analysis of the larger set of records, since the computed cluster includes clinical outcome prediction scores of individuals that are clinically more similar to the target individual that the remaining individuals.

Inclusion of clinical outcome prediction scores for the remaining individuals reduces the accuracy of the analysis. For example, for an elderly sick individual with multiple co-morbidities, inclusion of data from young health individuals skews the analysis.

The systems and/or methods and/or code instructions described herein generate new data in the form of the data structure that stores the computed clinical outcome prediction scores of the individuals, for example, the n-dimensional Euclidean space. The n-dimensional Euclidean space improves computational performance of a computing device in identifying the cluster of clinically significant individuals, by computing the nearest neighbors of a point in the n-dimensional Euclidean space representing the target individual.

The systems and/or methods and/or code instructions described herein improve an underlying process within the technical field of medical data, in particular, within the field of data mining of medical data.

The systems and/or methods and/or code instructions described herein do not simply describe the aggregation of data of members of a cluster of individuals clinically similar to the target individual, using a mathematical operation and receiving and storing data, but combine the acts of applying a trained classifier to the indication of medical tests results of a target individual (which are received over a network) to compute a set of clinical outcome prediction scores, computing a cluster of sets of clinical outcome prediction scores of individuals according to a requirement of a statistical distance with the set of clinical outcome prediction scores of the target individual, and outputting the aggregation for presentation by the client terminal. By this, the systems and/or methods and/or code instructions stored in a storage device executed by one or more processors described here go beyond the mere concept of simply retrieving and combining data using a computer.

The systems and/or methods and/or code instructions described herein are tied to physical real-life components, including one or more of: network equipment, physical user interfaces (e.g., display), a data storage device storing patient data, and a hardware processor(s) that execute code instructions.

Accordingly, the systems and/or methods and/or code instructions described herein are inextricably tied to computing technology and/or physical components to overcome an actual technical problem arising in analyzing medical data.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As used herein, the terms patient and individual are sometimes interchangeable.

As used herein, the phrase cluster of sets of clinical outcome prediction scores is sometimes interchangeable with the phrase cluster of individuals clinically similar to a target individual, since each set of clinical outcome predictions of the computed cluster is of a certain individual that represents clinically similarity to the target individual.

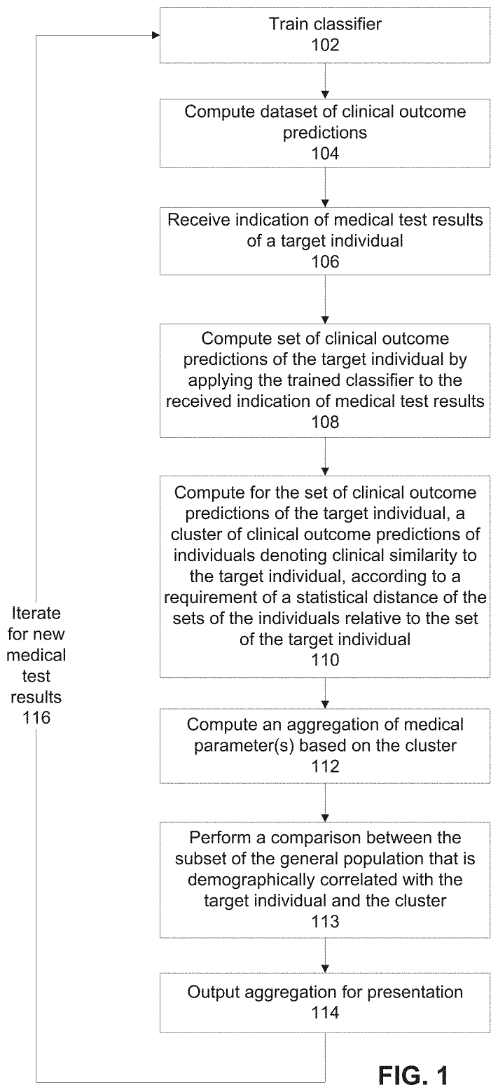

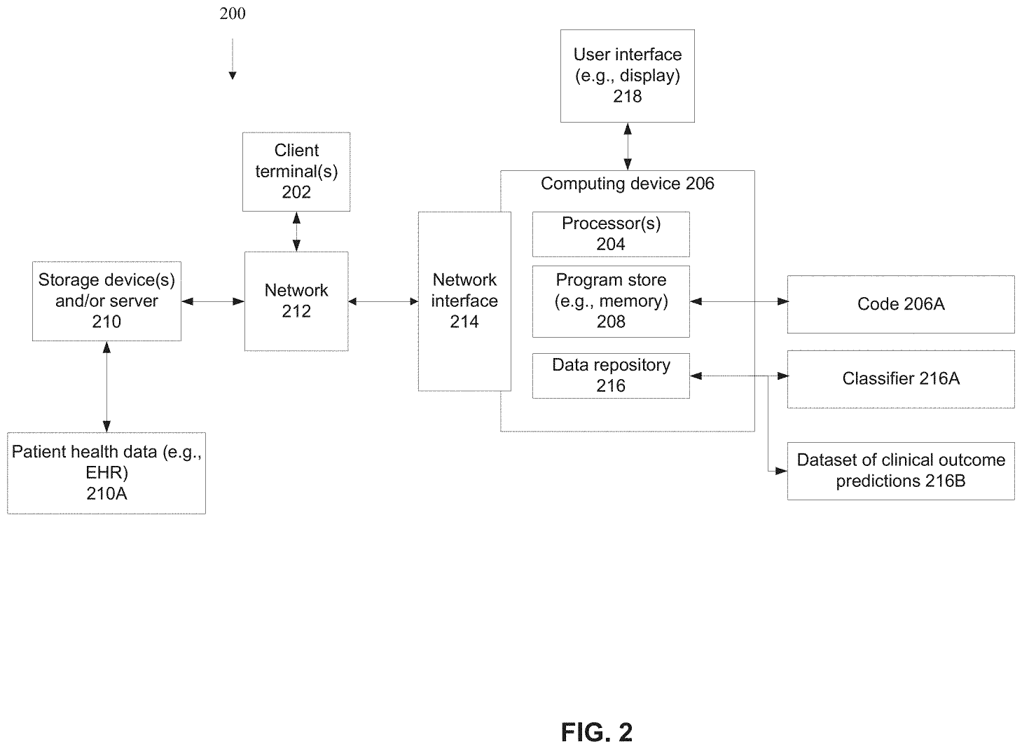

Reference is now made to FIG. 1, which is a flowchart of a method of providing a client terminal with an aggregation of at least one medical parameter of a cluster of individuals clinically similar to a target individual in response to an indication of current medical test results of the target individual, in accordance with some embodiments of the present invention. Reference is also made to FIG. 2, which is a block diagram of components of a system 200 for providing a client terminal 202 with an aggregation of at least one medical parameter of a cluster of individuals clinically similar to a target individual in response to an indication of current medical test results of the target individual, in accordance with some embodiments of the present invention. System 200 may implement the acts of the method described with reference to FIG. 1, by processor(s) 204 of a computing device 206 executing code instructions stored in a program store (e.g., memory) 208. Computing device 206 receives indications of medical test results, from client terminal 202, and/or from another computing device (e.g., laboratory information server (LIS) that stores the laboratory test results) and/or from an electronic health record (EHR) 210A hosted by a storage server 210 (and/or storage device).

The medical testing results may be provided via a network 212 connected to a network interface 214 of computing device 206. Exemplary network interfaces 214 include, for example, a network interface card, a wire connection, a wireless connection, other physical interface implementations, and/or virtual interfaces (e.g., software interface, application programming interface (API), software development kit (SDK)), a physical interface for connecting to a cable for network connectivity, network communication software providing higher layers of network connectivity, and/or other implementations. Network interface 214 may implement a healthcare communication protocol, for example, health level-7 (HL7).

Network 212 may include, for example one or more of: the internet, a local area network, a wireless network, a cellular network, a virtual private network, and a point to point connection with another computing device.

Computing device 206 may be integrated with an existing LIS (laboratory information systems) server and/or CDR (clinical data repository) server and/or PACS server(picture archiving and communication system) and/or EHR (electronic health record) storage server 210, for example, as a separate computing device that communicates with the LIS and/or EHR server over a network and/or via a direct connection, as code instructions that are stored in a data storage device of the LIS and/or EHR server and executed by processor(s) of the LIS and/or EHR server, and/or as a hardware unit that is integrated with LIS and/or EHR server, for example a hardware card or chip that is plugged into the hardware of the LIS and/or EHR server. Alternatively or additionally, computing device 206 may be implemented as, for example, a client terminal, a server, a computing cloud, a mobile device, a desktop computer, a thin client, a Smartphone, a Tablet computer, a laptop computer, a wearable computer, glasses computer, and a watch computer.

Computing device 206 may include locally stored software (e.g., code 208A) that performs one or more of the acts described with reference to FIG. 1, and/or may act as one or more servers (e.g., network server, web server, a computing cloud) that provides services (e.g., one or more of the acts described with reference to FIG. 1) to one or more client terminals 202 over network 214, for example, providing software as a service (SaaS) to the client terminal(s) 202, providing an application for local download to the client terminal(s) 202, and/or providing functions via a remote access session to the client terminals 202, for example, hosting a web site accessed via a web browser and/or application stored on client terminal 202. It is noted that computing device 206 may be provided as a turnkey solution provided by an external vendor, for example to client terminal(s) 202. Computing device 206 may be implemented as a block-chain based computer platform.

Client terminal(s) 202 accessing computing device 206 may include one or more of: a server, a computing cloud, a mobile device, a desktop computer, a thin client, a Smartphone, a Tablet computer, a laptop computer, a wearable computer, glasses computer, and a watch computer.

Processor(s) 204 of computing device 206 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 204 may include one or more processors (homogenous or heterogeneous), which may be arranged for parallel processing, as clusters and/or as one or more multi core processing units.

Storage device (also known herein as a program store, e.g., a memory) 208 stores code instructions implementable by processor(s) 204, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Storage device 208 stores code 208A that executes one or more acts of the method described with reference to FIG. 1.

Computing device 206 may include a data repository 216 for storing data, for example, a classifier 216A that stores the trained classifier that computes the set of clinical outcome prediction scores, and a dataset of clinical outcome prediction scores of a population of individuals (e.g., implemented as an n-dimensional Euclidean space) 216B. Data repository 216 may be implemented as, for example, a memory, a local hard-drive, a removable storage unit, an optical disk, a storage device, and/or as a remote server and/or computing cloud (e.g., accessed via a network connection).

Computing device 206 may connect via network interface 214 to network 212 (and/or another communication channel, such as through a direct link (e.g., cable, wireless) and/or indirect link (e.g., via an intermediary computing unit such as a server, and/or via a storage device) with one or more of:
Client terminal(s) 202, for example, when the client terminal 202 is used by a patient and/or physician to view the aggregation of medical parameter(s) of members of the cluster of individuals clinically similar to the target individual.
Server(s) 210 including an EHR server and/or the LIS server and/or CDR server that store and/or access patient medical data 210A, for example, from the EHR of the patient. The CDR may be implemented as a real time database that consolidates data from a variety of clinical sources to present a unified view for each patient. Data of individuals described herein may be obtained by computing device 206 from the data storage server 210 (e.g., EHR server and/or CDR server and/or LIS server).
Another external storage server that stores the computed trained classifier, and/or dataset of clinical outcomes prediction scores (e.g., n-dimensional Euclidean space).

Computing device 206 and/or client terminal(s) 202 include and/or are in communication with a user interface 218 that includes a mechanism for a user to enter data (e.g., provide the medical test results), and/or view presented data (e.g., the computed aggregation of the medical parameter(s) of the cluster of clinically similar individuals).

Exemplary user interfaces 218 include, for example, one or more of, a touchscreen, a display, a keyboard, a mouse, and voice activated software using speakers and microphone.

At 102, the classifier that receives indications of medical test results and computes a set of clinical outcome prediction scores is trained. Training may be performed by computing device 206, and/or by another computing device (e.g., remote server). The trained classifier may be stored in association with computing device 206, for example, as code instructions of trained classifier 216A stored in data repository 216.

The classifier may be trained according to the following exemplary method, which is not necessarily limiting:

One or more sets of indications of medical test results are, for example, extracted for patients from a medical database (e.g., EHR 210A, laboratory testing database, PACS server), obtained from output of devices that perform the test (e.g., wearable sensor, internet of things (IoT) medical device, laboratory measurement device), and/or from another computing device (e.g., mobile device, remote server). The medical testing results may be extracted for predefined medical tests, and/or for all available medical tests.

Exemplary medical tests include laboratory tests, physical exam findings, symptoms obtained from a medical history, radiological examination findings, and other medical tests and/or measurements performed by a medical device.

Exemplary laboratory tests include tests performed on samples of body fluids and/or tissue samples, for example, blood, urine, stool, phlegm, and cerebrospinal fluid. Exemplary laboratory tests measure one or more of the following: complete blood count (CBC), liver functions tests, kidney functions tests, electrolytes, GFR (globular filtration rate), potassium, sodium, cholesterol, triglycerides, ALKP, ALT, hemoglobin, HbA1C, blood glucose level (e.g., random glucose, glucose tolerance test), LDL, creatinine, ALT, blood sodium (Na) level.

Exemplary physical exam findings include: heart sounds, finger clubbing, cyanosis, mental status exam score, costovertebral angle tenderness, psoas sign, valgus.

Exemplary symptoms include: chest pain, abdominal pain, fatigue, dizziness, constipation, and difficulty swallowing.

Exemplary radiological examinations include findings obtained from one or more certain radiological examination. Exemplary radiological examination findings include: enlarged heart, splenomegaly, increased lung volume, consolidation, pericardial effusion, kidney stone, lympadenopahy, bowel dilation, degenerative bone changes.

Exemplary tests performed by a medical device include: electrocardiogram (ECG), cardiovascular stress test, Electromyogram (EMG), and blood pressure. The medical devices may be standalone medical devices, and/or internet of things (IoT) sensors and/or IoT devices.

The medical tests may be extracted for all patients, or for subset of patients, and/or a set of patient data may be excluded (e.g., outliers). The date (and/or time) of the medical test is termed herein prediction date.

Optionally, indication of determinants of health data may be extracted for the patients and/or target individual, and added to the medical test data for processing as described herein. The indication of health data may include demographic data (e.g., age, gender, income, geographic location), genetic data (e.g., inherited conditions, family history, genes associated with an increased risk of disease), nutrition (e.g., diet), and environmental exposure (e.g., exposure to population).

A diagnosis of a clinical outcome is extracted for the patients for whom the medical test results were extracted. The diagnosis of clinical outcome may be extracted, for example, from the EHR, from a billing database, and/or from a registry indicating a clinical condition of the patient. The diagnosis may be extracted based on one or more codes, optionally internationally recognized diagnostic codes. For example, codes indicative of renal failure include G_K05, G_1Z12, and G_1Z13. All available diagnoses may be extracted, or a predefined set of diagnosis may be extracted. Exemplary clinical outcomes each indicative of a certain pathology of a certain physiological system include death, neoplasm, ischemic heart disease, type two diabetes, liver disease, chronic renal failure, chronic obstructive pulmonary disease, acquired hypothyroidism, epilepsy, migraine, chronic fatigue syndrome, trigeminal neuralgia, hypertensive disease, nervous system disease, acute sinusitis, bell facial palsy, carpal tunnel syndrome, retinal detachment, diabetic retinopathy, degeneration of macula, glaucoma, vertiginous syndromes, hyperthyroidism, acute renal failure, heart valve disorders, haematuria, psoriasis, systemic lupus erythematosus, polymyalgia rheumatic, pulmonary embolism, cataract, cardiac dysrhythmias, and osteoporosis.

The date (and/or time) of the diagnosis is termed herein positive outcome dates. It is noted that the term positive does not necessarily mean a positive diagnosis, but is intended to represent some sort of diagnosis of the patients. Patients with a diagnosis are termed herein positive patients. Patients for which medical test results were extracted, but for which diagnosis of one or more clinical outcomes were not obtained are termed herein as negative patients.

For positive patients, members of the set of indications of medical test results of dates (i.e., prediction dates) that are outside of a defined time interval relative to the date of the diagnosis of clinical outcome (i.e., positive outcome date) are filtered out. The predefined time interval is selected to increase the correlation between the medical test results and the clinical outcome. Exemplary predefined time intervals include: about 1 year, about 3 years, and about 5 years. For example, blood tests within 3 years of a cancer diagnosis are stored, while blood tests older than 3 years from the cancer diagnosis are excluded. The remaining sets of medical test results are termed herein positive samples.

For negative individuals, members of the set of indications of medical test results of dates that are within the defined time interval relative to the date of computation of the trained classifier, and/or relative to the date at which the patient died and/or left the database are filtered out. Since there is no diagnosis, medical tests within the predefined time interval cannot accurately be said to be correlated with anything, while medical tests outside of the predefined time interval may be assumed to be correlated with no diagnosis. For example, for healthy patients with no diagnosis, blood tests older than 3 years are assumed to correlate with a healthy state and/or with no disease diagnosis.

A training dataset is created (e.g., stored as a file). The training dataset may be termed herein training outcome set. One set of patient records may be selected for training of the classifier, and the remaining set of patient records selected for creation of a validation set for validation of the classifier, for example, 70% of the patient records are designated for training of the classifier and the other 30% of the patient records are designated for validation of the classifier. The training dataset may be created by sampling a defined ratio of positive patients to negative patients. For example, a ratio of 7 negative patients for every positive patient. The validation dataset may be created based on the available prediction dates for the designated set of patients.

Optionally, for each patient included in the training and/or validation sets, additional data is extracted from a medical record of the respective patient, for example, from the patient's EHR 210A. The additional data is added to the training and/or validation datasets. The additional data may include demographic parameters, for example, age, and gender. The additional data may include smoking status, smoking history, recreational drug use, and recreational drug use history. The additional data may include prescribed medications that the patient is currently taking, and/or prescribed medications that the patient took during the last predefined time interval (e.g., 1, 3, 5 years) and which the patient stopped taking.

Optionally, trends and/or patterns are computed for the medical tests, optionally for the analytes measured in the medical tests. The trends and/or patterns are computed for one or more predefined time interval (e.g., the last 3 years, and the last year) relative to the diagnosis and/or relative to the date of computation of the classifier. Exemplary computed trends and/or patterns include: last value, slope (of line computed between two data points and/or of regression line fitted to the data points), minimum value, and maximum value. The trends and/or patterns may be included in the training and/or validation dataset.

The training and/or validation dataset may be implemented as a feature matrix.

Optionally outliers are removed, for example having a value above a predefined number of standard deviations from the means, for example, greater than 15 standard deviations.

The classifier is trained using the training dataset and/or feature matrix. The classifier may be validated using the validation dataset.

Optionally, the trained classifier is implemented as a gradient boosting classifier. The gradient boosting classifier may be implemented as an XGBOOST classifier. The XGBOOST classifier may be trained using the following exemplary parameters (which may be adjusted accordingly): booster=gbtree; objective=binary:logistic; eta=0.05; gamma=1; max_depth=5; num_round=300; min_child_weight=6.

It is noted that XGBOOST is exemplary. Other classifiers that perform according to a performance requirement may be selected. For example, in performance testing, inventors discovered that the XGBOOST classifier outperformed other machine learning methods such as random forest.

Optionally, another classifier is trained based only on the additional data, optionally the demographic data (e.g., age, gender), optionally from the feature matrix. The other classifier may be implemented as, for example, linear regression. The other classifier is referred to herein as a demographic classifier. The demographic classifier computes the clinical outcome predictors from demographic data.

Optionally, the trained classifier outputs a computed score per clinical outcome per patient. The score may be indicative of the computed probability of the patient developing the clinical outcome within the upcoming predefined interval (e.g., within the next 1, 3, or 5 years). The score of the clinical outcome may be associated with a weight, for example, relatively more serious medical conditions (e.g., cancer, death), are associate with relatively larger weights in comparison to relatively less serious medical conditions (e.g., trigeminal neuralgia).

At 104, the dataset storing the set of clinical outcome prediction scores for the individuals having records in the database (e.g., EHR 210A) is computed. The dataset of clinical outcomes may be stored as dataset 216B by data repository 216 of computing device 206.

The dataset may be created based on the validation dataset (as described with reference to act 102), the training dataset, and/or the full dataset.

The dataset is created by applying the trained classifier on the data (i.e., the medical test results and/or additional clinical data) for each of the individuals (i.e., on the validation dataset, training dataset, and/or full dataset) to compute the clinical outcome prediction scores for each of the individuals in the dataset.

The dataset includes the computed clinical outcome prediction score, optionally as a score for each clinical outcome, and may include the additional data (e.g., age, gender). The dataset may be implemented as a vector, a matrix, comma separated fields, or other data structures.

Optionally, the data stored in the dataset is standardized, for example, relative to a mean assigned a value of zero, and coordinates defining standard deviations from the mean (e.g., one standard deviation per coordinate unit).

The dataset of clinical outcome prediction scores may be arranged as an n-dimensional Euclidean space. Each of the n dimensions denotes a respective clinical outcome prediction. A point for each patient is located in the n-dimensional space according to the value of the score computed for each clinical outcome prediction by the classifier.

At 106, an indication of medical test results of the target individual is received by computing device 206. The indication of medical test results is received from a client terminal, for example, manually entered by a user, accessed from the EHR of the patient, and/or transmitted by a LIS server.

The medical test results may be based on a predefined list of tests which the individual is set to perform for the purpose of computing the set of clinical outcome prediction scores. Alternatively, the medical test results are based on tests that the individual underwent for other purposes, and are transmitted to the computing device for further processing to compute the set of clinical outcome prediction scores.

At 108, the trained classifier is applied to the received indication of medical test results of the target individual to compute a set of clinical outcome prediction scores.

Optionally, the trained classifier computes a prediction score for each member of the set of clinical outcome predictions each indicative of a certain pathology of a certain physiological system of the respective individual.

At 110, a cluster of sets of clinical outcome prediction scores for individuals in the dataset is computed for a set of clinical outcome predictions each indicative of a certain pathology of a certain physiological system of the respective individual. The computed cluster of sets of clinical outcome prediction scores denote a cluster of individuals clinically similar to the target individual.

The cluster is computed according to a requirement of a statistical distance between the set of computed scores of the clinical outcome predictions of the individuals of the dataset and the set of computed scores of the clinical outcome predictions of the target individual.

The statistical distance requirement may denote, for example, a predefined number of the sets of clinical outcome prediction scores of individuals (e.g., 1000) that have the smallest statistical distance to the set of clinical outcome prediction scores of the target individual, and/or a maximum statistical distance to the furthest set of clinical outcome prediction scores of a certain individual, and/or a statistical distance threshold value. The statistical distance may be computed according to weights of the n-dimensions and/or axes. Optionally, the set of clinical outcome prediction scores computed for the target individual is mapped as a point in the n-dimensional Euclidean space.

The cluster is identified by computing the nearest neighbors of the mapped point of the target individual, according to the statistical distance requirement. The statistical distance requirement may define a predefined number of nearest neighbors according to distances between the mapped set of clinical outcome prediction score for the target individuals and the location of each of the individuals in the n-dimensional Euclidean space, for example, the 1000 closest neighbors to the point of the target individual.

In another implementation, a value is computed for each individual as an aggregation (e.g., weighted sum and/or weighted average) of the scores of the clinical outcome predictions of the respective individual. The statistical distance requirement may be defined as the aggregated values of the scores of the individuals that are closest to the aggregated value of the scores of the target individual.

Optionally, the cluster is computed based on a k-nearest neighbor (KNN) method, and/or other clustering methods, and/or other statistical distance computation methods.

The nearest neighbors may be selected with the following constraints: the target individual cannot be its own neighbor, and the closest prediction date is selected for each patient such that the same patient is prevent from being identified as a neighbor multiple times (prevent over-sampling).

Optionally, the prevalence of each clinical outcome prediction in the cluster is computed, referred to herein as cluster prediction.

Optionally, a calibration is performed. A calibration set is designated, for example, as a predefined percentage of the available number of individuals having records stored in the medical database (e.g., EHR 210A), for example, 10% of the individuals.

The calibration may be performed by computing the clinical outcome prediction scores by applying the classifier (e.g., gradient boosting classifier) applied to the calibration set, and/or another set of clinical outcome prediction scores by applying the demographic classifier applied to the calibration set.

The prevalence of each clinical outcome prediction in the calibration cluster is computed. The computed clinical prediction outcomes for the calibration set (optionally including the scores) are sorted and may be divided into bins (e.g., of a selected size). Each bin is calibrated according to the prevalence of the clinical prediction outcomes in the respective bin. It is noted that even when the cluster denotes pre-calibrated clinical outcome predictions (i.e., the cluster prediction), the re-calibration described herein may be performed.

The re-calibration ignores statistically insignificant results, for example, clinical outcome prediction scores based on only the closest predefined number of individuals may vary significantly.

Optionally, the cluster is analyzed to discern valid results from statistical noise. For example, computing the Fisher statistical significance of the prevalence of each clinical outcome prediction indicative of a certain pathology of each certain physiological system of the cluster relative to the clinical prediction outcomes of the demographic classifier applied to the medical results of the target individual.

Clinical outcome predictions with Fisher p-values below a requirement (e.g., threshold, for example, 0.005) are maintained (i.e. clinical outcome predictions with Fisher p-values above a requirement are excluded) and/or reported and/or further processed. The remaining clinical outcome prediction scores denote a risk of the target individual developing the clinical outcome prediction. In another example, the ratio of the prevalence of clinical outcome predictions of the cluster relative to clinical outcome predictions computed by the demographic classifier, is computed.

Clinical outcome prediction scores having a computed ratio above a predefined value (e.g., greater than 150%) are maintained and/or reported and/or further processed (i.e., clinical outcome prediction scores having a computed ratio below the predefined value are excluded). The remaining clinical outcome prediction scores denote an elevated risk of the target individual developing the clinical outcome prediction. In yet another example, clinical outcome predictions with a prediction score value above a requirement (e.g., threshold) are identified, for example, above 0.01%. The remaining clinical outcome prediction scores denote an absolute risk of the target individual developing the clinical outcome prediction.

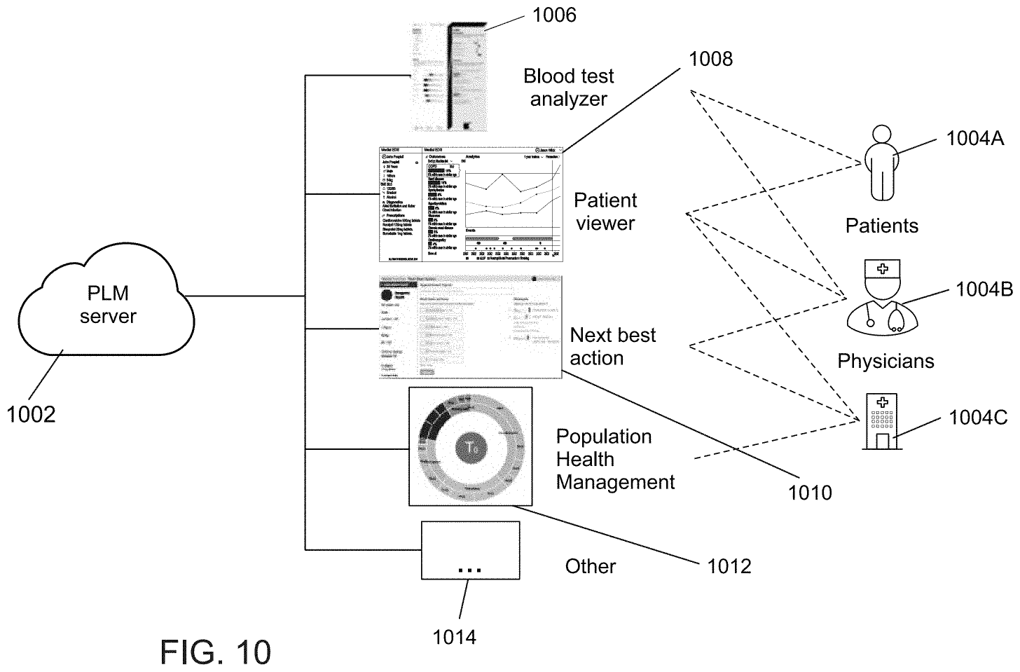

Reference is now made to FIG. 11, which is a graph depicting computation of 1000 individuals clinically similar to the target individual, in accordance with some embodiments of the present invention. The 1000 individuals are identified according to computed sets of 25 clinical outcome prediction scores.

X-axis 1102 denotes 25 clinical outcome prediction scores for a future time frame of the next 330 days (about 1 year), and predicted for the next 330-660 days (about the second year). A denotes chronic renal failure. B denote death. C denotes diabetes type II. D denotes gout. E denotes leukemia. F denotes neoplasm. G denotes ischemic heart disease. H denotes liver disease. I denotes chronic obstructive pulmonary disease. J denotes diabetic retinopathy. K denotes degeneration of macula and posterior pole. L denotes osteoporosis. M denotes hyperthyroidism. N denotes acquired hypothyroidism. O denotes hypertensive disease. P denotes heart failure. Q denotes intracerebral hemorrhage. R denotes cerebral infarction. S denotes peripheral vascular disease. T denote inflammatory bowel disease. U denotes cirrhosis of liver NOS. V denotes cholelithiasis. W denotes acute pancreatitis. X denotes pulmonary heart disease. Y denotes cerebrovascular disease. Z denotes lung cancer. A1 denotes digestive cancer. B1 denotes hematologic cancer. C1 denotes kidney cancer. D1 denotes lymphoma. Y axis 1104 denotes the area under the curve, computed per future prediction interval (i.e., the next 0-330 days, or the upcoming 330-660 days), per outcome, by aggregation of the clinical outcome scores computed for the 1000 individuals identified as clinically similar to the target individual.

Trend line 1106 denotes computation of the scores of the clinical outcome predictions by the demographic classifier. Trend line 1108 denotes computation of the scores of the clinical outcome predictions according to a naïve prediction based on age and gender. Trend line 1110 denotes computation of the scores of the clinical outcome predictions by the XGB classifier. Trend line 1112 denotes computation of the scores of the clinical outcome predictions by the XGB classifier and computation of the cluster based on the KNN method.

Referring now back to FIG. 1, at 112, an aggregation of one or more medical parameter of the cluster of clinically similar individuals is computed. The aggregation may include the clinical outcome predictions, for example, an average of the scores for each clinical outcome prediction of the cluster and/or a distribution of the scores for each clinical outcome prediction of the cluster.

The medical parameter may be defined, for example, by a user using a client terminal (e.g., via a graphical user interface) to manually select the medical parameter from a list of medical parameters, and/or from medical parameters stored in the medical database (e.g., EHR) of the patients of the cluster, and/or may be predefined as a configuration stored in a memory of the computing device.

The following are exemplary medical parameters. Other medical parameters may be defined accordingly:
The aggregation of the medical parameter(s) may be computed as a prevalence of one or more of the clinical outcome predictions for the cluster of clinically similar individuals. For example, when the clinical outcome predictions include type two diabetes and chronic obstructive pulmonary disease (COPD), the prevalence of diabetes and COPD in the cluster is computed. Optionally, the prevalence of the clinical outcome predictions computed for the cluster are computed for a general population. Optionally, the prevalence is computed for the subset of the general population that is demographically correlated with the target individual
The aggregation of the medical parameter(s) may be computed as an aggregated (e.g., average) age of members of the cluster of clinically similar individuals. The average age of members of the cluster of clinically similar individuals denotes a biological age of the target individual. For example, for a 40 year old individual being matched with a cluster where the average age is 60 years may indicate to the 40 year old that his/her body is similar to a 60 year old body.
The aggregated (e.g., average) age of members of the cluster that are most closely correlated with one or more clinical outcome predictions of the target individual is computed for each correlated clinical outcome prediction according to a requirement, for example, above a score threshold and/or individuals diagnosed with the disease. The computed average age denotes a biological age of the organ and/or organ system of the target individual of the clinical outcome prediction. For example, for a target individual having a significant score for COPD, the individuals having similar COPD scores (e.g., according to a requirement) are identified from the cluster. The age of the COPD correlated individuals is computed, and presented as a biological lung and/or respiratory system age. For example, for a 35 year old individual being matched with a cluster where the average COPD age is 65 years may indicate to the 35 year old that his/her lungs are similar to the lungs of a 65 year old.
The aggregation of the medical parameter(s) may include computing an incidence of a medical treatment and/or specialist consultation and/or prescribed medications for the cluster of clinically similar individuals. The incidence of the medical treatment and/or specialist consultation and/or prescribed medications may be computed for the general population and/or the subset of the general population demographically correlated with the target individual. Exemplary medical treatments include: invasive surgeries, minimally invasive surgeries, non-invasive treatments such as external application of energy (e.g., radiation, ultrasound), and implantation of a device (e.g., stent). Exemplary prescribed medications include oral delivered medicines (e.g., pills), implanted drug delivery devices, frequently administered medications (e.g., once a day pills), and one time administered medications (e.g., injection). Exemplary specialist consultations include: referrals by a general practitioner to a specialist in a certain medical field, self-initiated visit to the specialist, and a second opinion of another specialist in the same field as the first specialist consultation.

Optionally, a difference between the incidence of the medical treatment, the prescribed medication, and/or the specialist consultation computed for the cluster in comparison to the incidence of the medical treatment, the prescribed medication, and/or the specialist consultation computed for a general population that is demographically correlated with the target individual. An indication of the medical treatment, the prescribed medication, and/or the specialist consultation is generated when the incidence of the medical treatment, the prescribed medication, and/or the specialist consultation is statistically significant between the cluster and the general population.

Alternatively or additionally, the aggregation of the medical parameter includes computing for an indication of the medical treatment, the prescribed medication, and/or the specialist consultation, the effect of the contribution of the medical treatment, the prescribed medication, and/or the specialist consultation in a difference between a prevalence of at least one clinical outcome prediction computed for the cluster in comparison to the prevalence of the at least one clinical outcome prediction computed for the general population that is demographically correlated with the target individual. An indication of the at least one clinical outcome prediction and the medical treatment, the prescribed medication, and/or the specialist consultation when the effect of the contribution on the prevalence of the at least one clinical outcome prediction is statistically significant between the cluster and the general population.
The aggregation of the medical parameter(s) may include computing for members of the cluster of clinically similar individuals, an aggregation (e.g., average, distribution) of a value of an analyte of the indication of laboratory test results of the target individual.

Optionally, at 113, a subset of the general population that is demographically correlated with the target population is compared to the cluster.

The aggregation of the one or more medical parameters is computed for the subset of the general population that is demographically correlated with the target individual, for example, the same gender and/or a common age (e.g. within a tolerance requirement, for example, within a range of +/−5 years from the age of the target individual). The aggregation of the one or more medical parameters computed for the cluster may be compared to the aggregation of the one or more medical parameters computed for the general population and/or the subset of the general population. The comparison is performed, for example, to identify increased risk or decreased risk of the one or more medical parameters between the cluster and the subset of the general population that is demographically correlated with the target individual, to identify increase or decrease prevalence of specialist consultations and/or medical treatments and/or prescriptions.

Optionally, the comparison between the subset of the general population that is demographically correlated with the target population and the cluster is performed with respect to the aggregation (e.g., average, distribution) of the value of one or more analytes of the indication of laboratory test results of the target individual. The distribution of the value of the analyte may be computed for the general population and/or the sub-set of the general population that is demographically correlated with the target individual. For example, for the target individual that had a blood test in which sodium levels were measured, the average sodium level is computed for the cluster, and the distribution of sodium levels is computed for the sub-set of the general population.

Alternatively or additionally, for a value (optionally a range of values) and/or a trend of values of an analyte(s) included in the indication of laboratory test of the medical test results of the target individual, a computation is performed to determine the effect of the contribution of the value (e.g., range) and/or the trend of values of the analyte in a statistically significant difference between a prevalence of clinical outcome prediction(s) computed for members of the cluster having similar value(s) and/or trend(s) of the analyte in comparison to the prevalence of the clinical outcome prediction(s) computed for the general population that is demographically correlated with the target individual. An indication (e.g., message, metadata, presentation) of the value (e.g., range) and/or trend of the analyte is generated when the effect of the contribution on the prevalence of the clinical outcome prediction(s) is statistically significant between the members of the cluster with similar value(s) and/or trend(s) of the analyte(s) and the general population (optionally the demographically correlated sub-population).

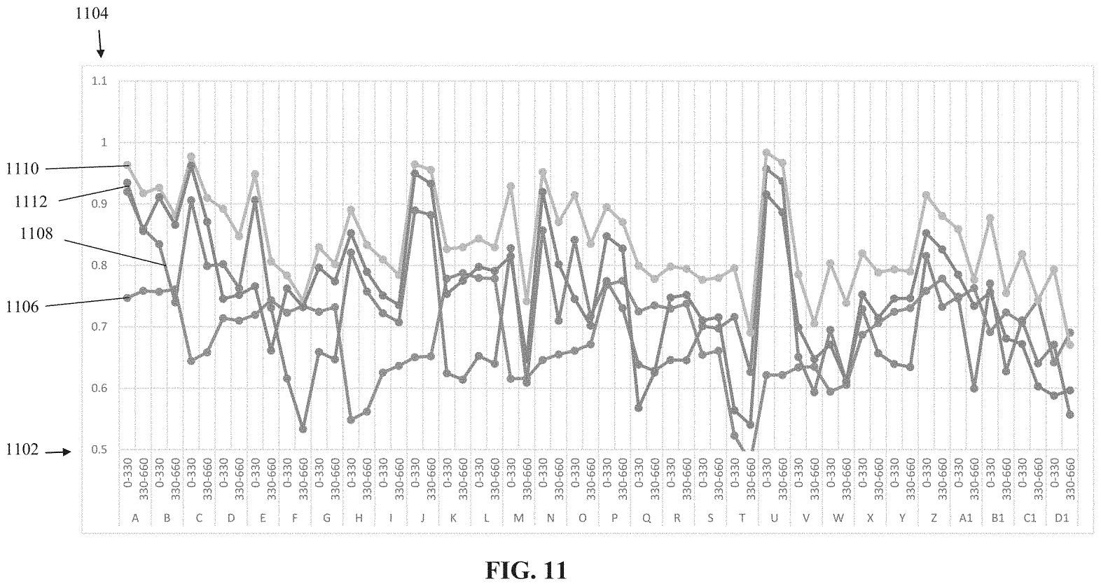

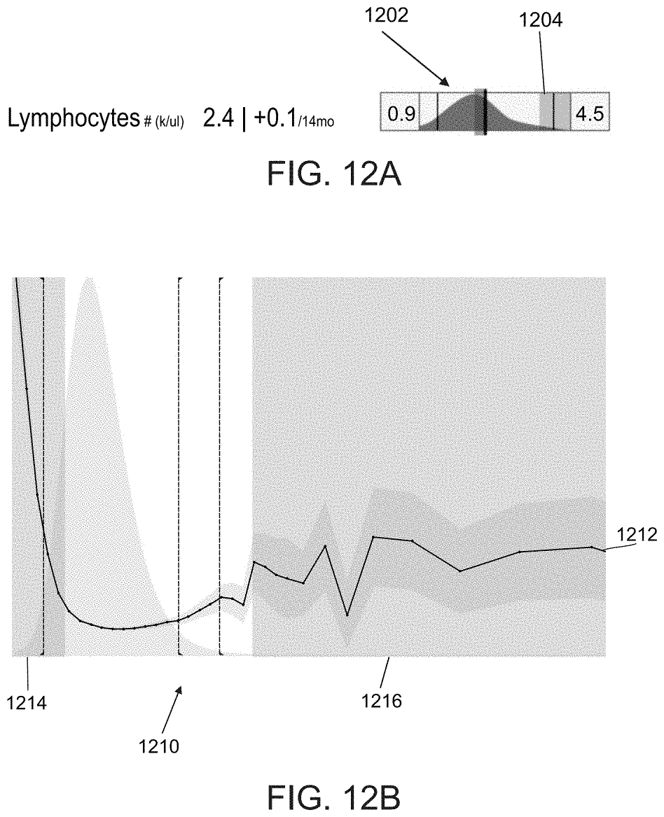

Reference is now made to FIG. 12A, which is an exemplary presentation (e.g., displayed on a display within a GUI) of a graph 1202 of lymphocyte values, indicating a region 1204 of values for which members of the cluster having lymphocyte values falling within region 1204 have a statistically significant difference in comparison to the general population (optionally the demographically correlated sub-population) in terms of the prevalence of one or more clinical outcome predictions, for example, mortality, cancer, or an index of general health computed from multiple clinical outcome predictions (optionally weighted) in accordance with some embodiments of the present invention.

The value (e.g., range) and/or trend of the analyte for which the contribution on the prevalence of the clinical outcome prediction(s) is statistically significant between the members of the cluster with similar value(s) and/or trend(s) of the analyte(s) and the general population (optionally the demographically correlated sub-population) may be computed

Reference is now made to FIG. 12B, which is an example of a graph 1210 for an exemplary computation of the value (e.g., range) and/or trend of the analyte for which the prevalence of the clinical outcome prediction(s) is statistically significant between the members of the cluster with similar value(s) and/or trend(s) of the analyte(s) and the general population, in accordance with some embodiments of the present invention. Graph 1210 is a plot 1212 of lymphocyte values versus a clinical outcome prediction (mortality score for this exemplary case), for which regions 1214 and 1216 denote ranges of lymphocyte values for which the mortality is statistically significant between the members of the cluster and the general population (e.g., lift >1.5). At 114, the aggregation is outputted for one or more of: presentation by the client terminal, presentation on another display of another client terminal, forwarding to a remote server, local stored, and/or for further processing. The aggregation may be presented within a graphical user interface (GUI) displayed on the display of the client terminal.

Optionally, the computed biological age is provided for presentation on the client terminal.

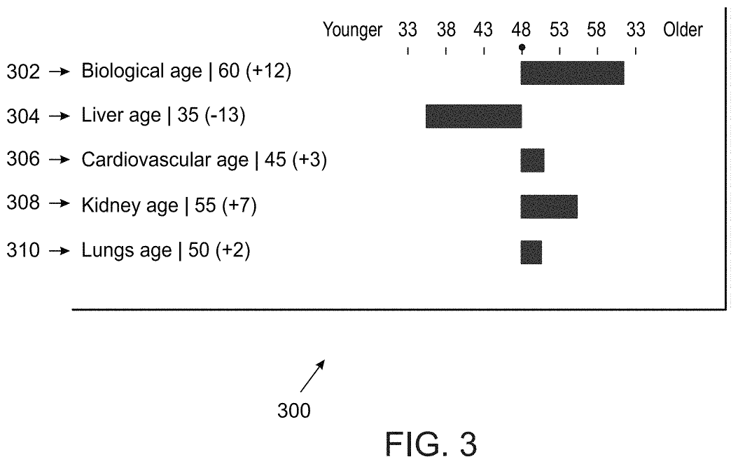

Reference is now made to FIG. 3, which is a schematic of an exemplary GUI 300 presenting computation of the biological age of the target individual based on an aggregation of the age of members of the cluster, in accordance with some embodiments of the present invention. A biological age 302 of the target individual is computed based on the aggregated age (e.g., average) of the members of the cluster. For example, GUI 300 depicts that the age of the target individual is 48, while the computed biological age 302 is 32. Liver age 304 denotes the aggregated age (e.g., average) of the members of the cluster associated with one or more clinical outcome predictions associated with liver disease. For example, GUI 300 depicts that the computed liver age is 35, which is 13 years less than the age of the target individual. Cardiovascular age 306, kidney age 308, and lung age 310 are computed accordingly.

Optionally, the prevalence of one or more clinical outcome predictions computed for the cluster and optionally computed for the demographically correlated subset of the general population are provided for presentation on the client terminal. Clinical outcome predictions computed for the cluster having a prevalence that is not statistically different from the prevalence computed for the demographically correlated subset of the general population may not necessarily be presented. Clinical outcome predictions with a prevalence that is greater and/or smaller than the prevalence computed for the demographically correlated subset of the general population are presented.

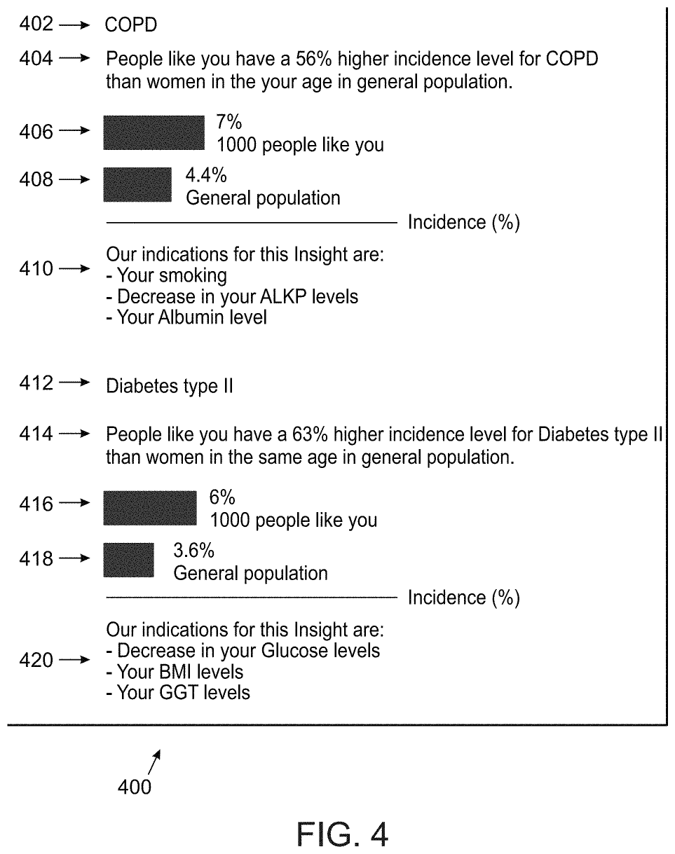

Reference is now made to FIG. 4, which is a schematic of an exemplary GUI 400 presenting computation of the prevalence of clinical outcome(s) of the cluster statistically significantly different from clinical outcomes of a demographically correlated subset of the general population, in accordance with some embodiments of the present invention. COPD 402 is identified as a clinical outcome of the cluster statistically significantly different from clinical outcomes of a demographically correlated subset of the general population. GUI 400 provides a textual summary 404 “People like you have a 56% higher incidence for COPD than women in your age in general population”. Bar graph 406 indicates a COPD incidence of 7% in the cluster of 1000 individuals, and bar graph 408 indicates a COPD incidence of 4.4% in the general demographically correlated population. Text message 410 indicates the clinical data (i.e., smoking status) and the blood medical test results (i.e., decrease in ALKP level and Albumin level) which were processed to compute the COPD incidence (i.e., the medical test results and/or clinical data to which the classifier was applied for computing the point in the n-dimensional space). Similarly, Diabetes type II 412 is identified as a clinical outcome of the cluster statistically significantly different from clinical outcomes of a demographically correlated subset of the general population. GUI 400 provides a textual summary 414 “People like you have a 63% higher incidence for Diabetes type II than women in the same age in general population”. Bar graph 416 indicates a Diabetes incidence of 6% in the cluster of 1000 individuals, and bar graph 418 indicates a Diabetes incidence of 3.6% in the general demographically correlated population. Text message 420 indicates the clinical data (i.e., BMI level) and the blood medical test results (i.e., decrease in Glucose level and GGT level) which were processed to compute the Diabetes incidence.

Alternatively or additionally, the incidence of the medical treatment and/or specialist consultation and/or prescribed medications for the cluster and optionally for the demographically correlated general population is provided for presentation by the client terminal. Medical treatment and/or specialist consultation and/or prescribed medications identified for the cluster having an incidence that is not statistically different from the incidence computed for the demographically correlated subset of the general population may not necessarily be presented.

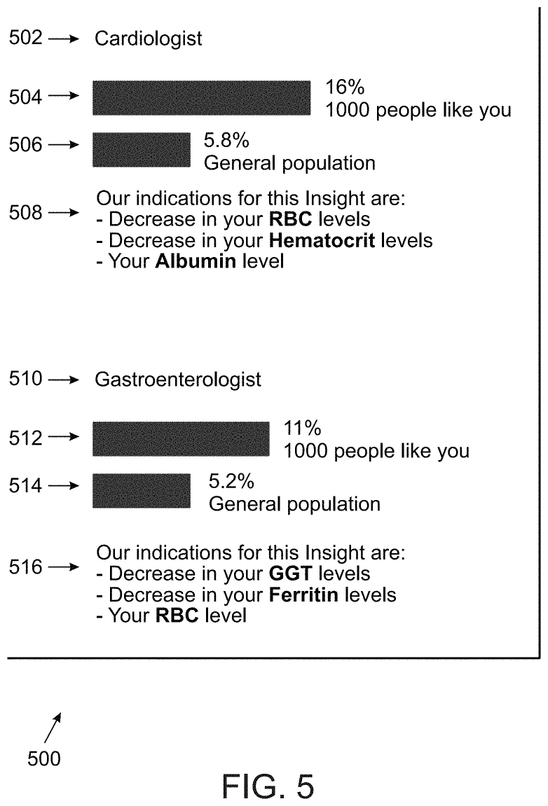

Reference is now made to FIG. 5, which is a schematic of an exemplary GUI 500 presenting computation of the incidence of specialist consultations of the cluster that are statistically significantly different from specialist consultations of a demographically correlated subset of the general population, in accordance with some embodiments of the present invention. A cardiologist consultation 502 is identified as a specialist consultation of the cluster that is statistically significantly different from specialist consultations of a demographically correlated subset of the general population. Bar graph 504 indicates an incidence of a cardiologist visit of 16% in the cluster of 1000 individuals, and bar graph 506 indicates an incidence of a cardiologist visit of 5.8% in the general demographically correlated population. Text message 508 indicates the blood medical test results (i.e., decrease in RBC level, decrease in Hematocrit level, and Albumin level) which were processed to compute the cardiologist consultation incidence. A gastroenterologist consultation 510 is identified as a specialist consultation of the cluster that is statistically significantly different from specialist consultations of a demographically correlated subset of the general population. Bar graph 512 indicates an incidence of a gastroenterologist visit of 11% in the cluster of 1000 individuals, and bar graph 514 indicates an incidence of a gastroenterologist visit of 5.2% in the general demographically correlated population. Text message 516 indicates the blood medical test results (i.e., decrease in GGT level, decrease in Ferritin level, and RBC level) which were processed to compute the gastroenterologist consultation incidence.

Alternatively or additionally, the value of the analyte of the target individual, the aggregated (e.g., average) value of the analyte for the cluster of clinically similar individuals, and/or the distribution of the value of the analyte of the general population and/or subset of the general population demographically correlated with the target individual, are provided for presentation by the client terminal.

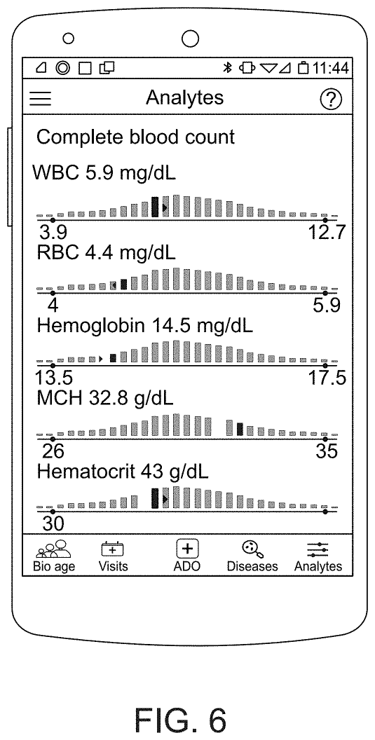

Reference is now made to FIG. 6, which is a schematic depicting an application 600 running on a smartphone displaying the aggregated (e.g., average) value computed from the cluster, for each analyte measured by the medical tests obtained for the target individual, relative to a distribution of the value of the analyte in the general population demographically correlated with the target individual, in accordance with some embodiments of the present invention.

At 116, acts 102-114 may be automatically iterated, for example, for each medical test ordered for the patient for which new medical test results are received. The iteration of the acts updates the computation of the cluster. When a statistically significant change in the aggregated value(s) (e.g., increased risk of clinical outcome(s), increased incidence of specialist consultation and/or medical intervention) is identified for the cluster relative to the general population (optionally the demographically correlated sub-population), an alert may be automatically transmitted to the client terminal and/or presented within a GUI.

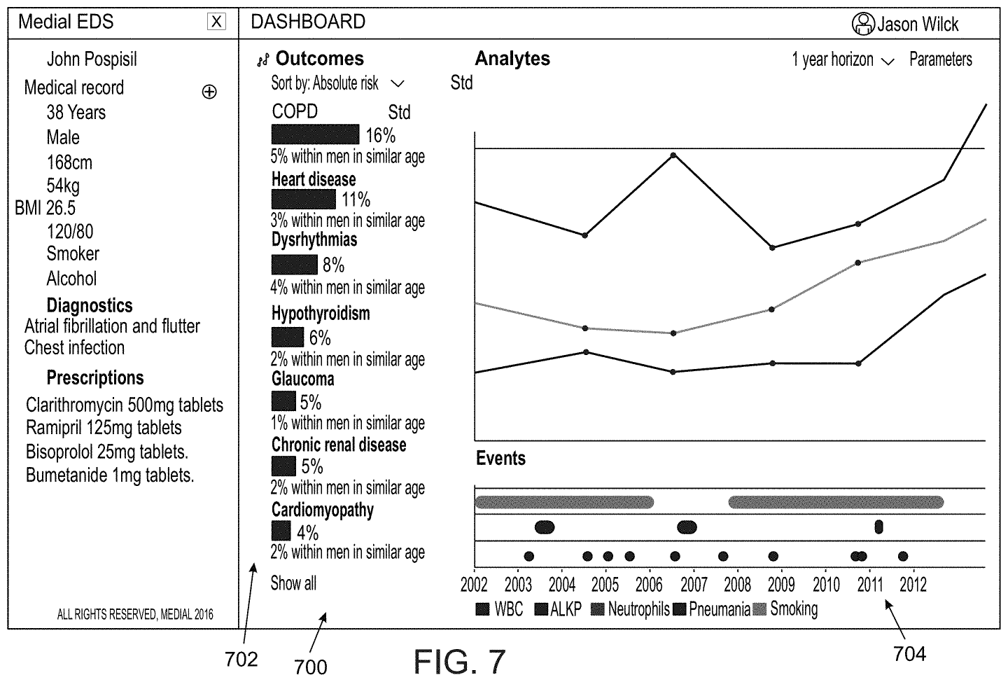

Reference is now made to FIG. 7, which is a schematic of an exemplary GUI 700 that presents prevalence of clinical outcome predictions 702 in association with a graph 704 depicting trends in values of analytes measured in laboratory tests and/or changes to medical data (e.g., smoking status), and medical history 706 (e.g., demographic information, medical diagnoses, and prescriptions), in accordance with some embodiments of the present invention.

Optionally, members of the cluster and/or physicians treating members of the cluster may be invited to join and/or automatically joined to a social network. Members of the social network, which are clinically similar (and/or treat clinically similar patients) may share information.

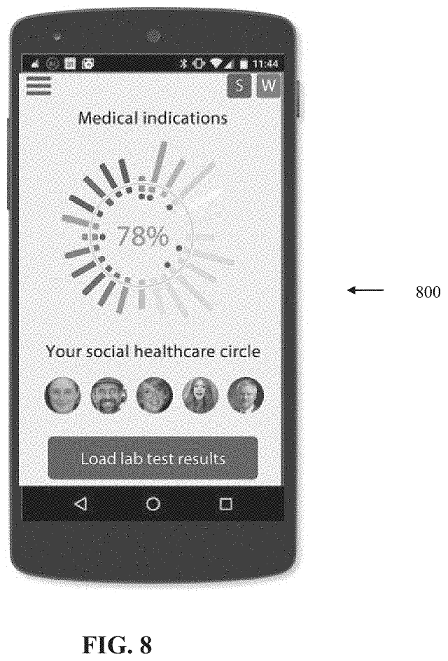

Reference is now made to FIG. 8, which is a schematic depicting a social network application 800 displayed on a smartphone for clinically similar individuals, in accordance with some embodiments of the present invention.

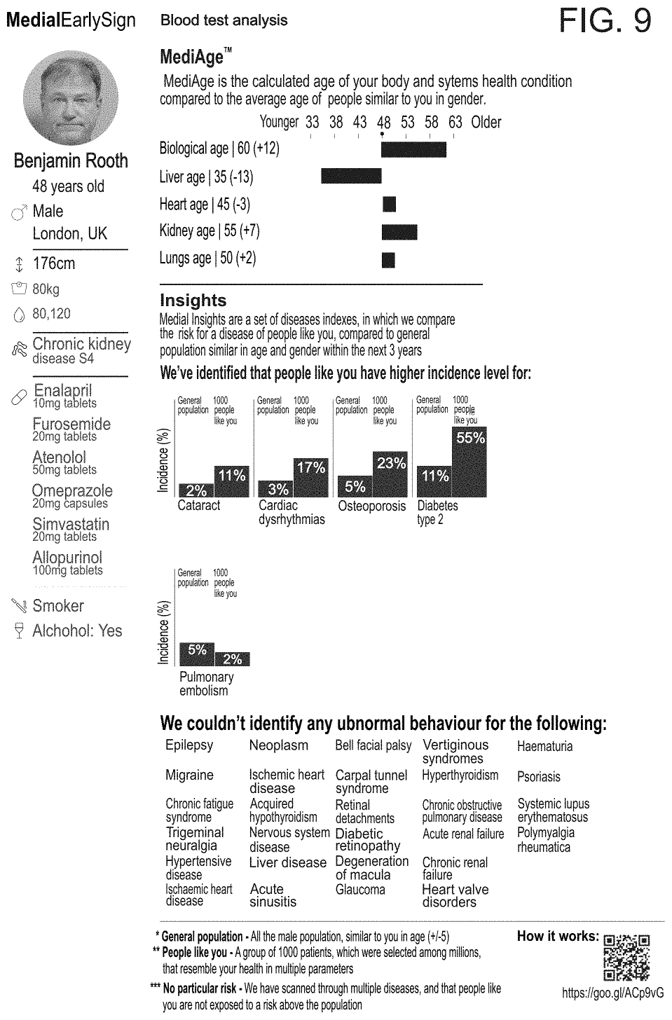

Reference is now made to FIG. 9, which is a schematic depicting a report and/or GUI presenting a summary of multiple aggregations computed based on the identified cluster, according to the medical test results of the target individual, in accordance with some embodiments of the present invention. The report and/or GUI of FIG. 9 include one or more of: computed biological age, computed organ and/or physiological system age, clinical outcomes with statistically significant higher incidence compared to the general population (optionally the demographically correlation population), clinical outcomes with statistically significant lower incidence compared to the general population (optionally the demographically correlation population), list of possible clinical outcomes, and blood test results in which the aggregated value computed for the cluster is compared to a distribution of values in the general population (optionally the demographically correlation population).

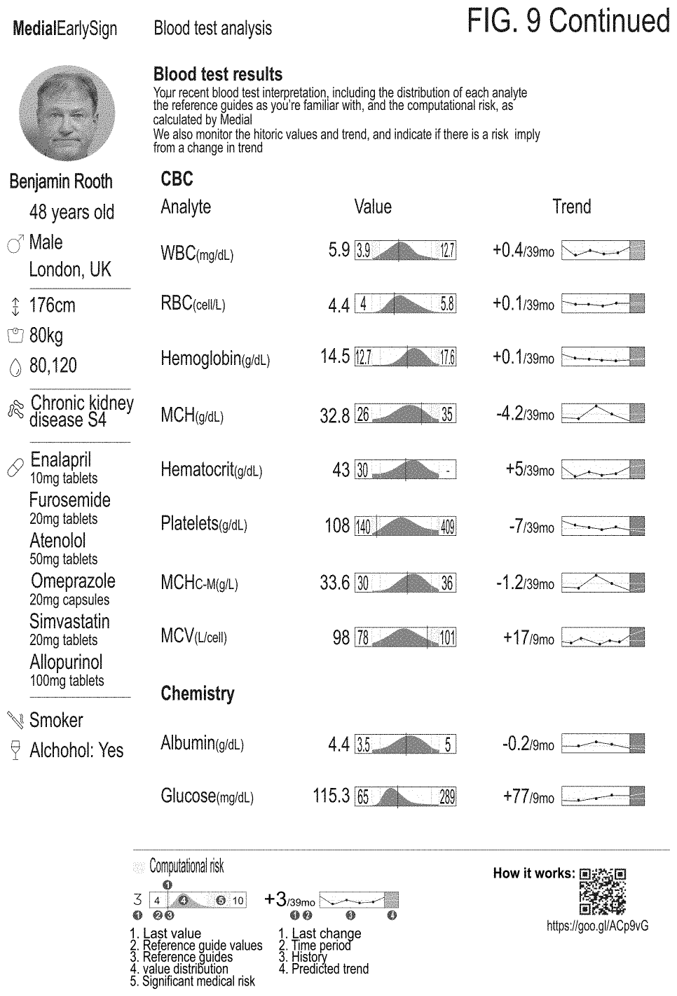

Reference is now made to FIG. 10, which is a schematic depicting applications provided by server 1002 to client terminals of users (e.g., patients 1004A, physicians 1004B, and medical facilities 1004C) over a network, based on aggregation of data of a cluster of individuals clinically similar to a target individual according to medical test results of the target individual, in accordance with some embodiments of the present invention. The applications may be provided by common GUI and/or common application based on different selections, and/or may be accessed as independent applications. Exemplary applications include:
A blood test analyzer 1006 that presents the aggregated (e.g., average) value of blood tests of the cluster, as described herein.
A patient viewer application 1008 that presents prevalence for clinical outcome predictions of the cluster that are statistically significantly different from prevalence for clinical outcome predictions of the general population.
A next best action application 1010 that presents incidence of specialist consultations and/or medical treatments and/or prescribed medications of the cluster that are statistically significantly different from incidence of specialist consultations and/or medical treatments and/or prescribed medications for clinical outcome predictions of the general population.
A population health management application 1012 that presents aggregation of data of the cluster.
Customized applications 1014 based on aggregation of custom defined medical parameters of the cluster.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant computing devices will be developed and the scope of the term computing device is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of providing a client terminal with an aggregation of at least one medical parameter of members of a cluster of individuals clinically similar to a target individual, comprising:
receiving by a computing system in communication with a dataset storing a set of computed clinical outcome prediction scores for each of a plurality of individuals, via a network, an indication of medical test results of the target individual;
applying a trained classifier to the indication of medical test results to compute, for the target individual, a set of clinical outcome prediction scores, wherein each respective score is indicative of a prediction of a certain pathology of a plurality of pathologies of a certain physiological system of a plurality of physiological systems of the target individual;
computing for the set of clinical outcome prediction scores of the target individual, a cluster of sets of computed clinical output predictions stored by the dataset, wherein the cluster of sets of computed clinical outcome prediction scores is computed according to a requirement of a statistical distance of sets of computed clinical outcome prediction scores stored by the dataset relative to the set of clinical outcome prediction scores of the target individual,
wherein each set of clinical outcome prediction scores is indicative of a predicted co-morbidity of a plurality of pathologies of a plurality of physiological systems,
wherein the individuals defined by the computed cluster denote individuals clinically similar to the target individual in terms of predicted co-morbidity of the plurality of pathologies of the plurality of physiological systems;
computing an aggregation of at least one medical parameter of the cluster; and
outputting the aggregation for presentation by the client terminal.

2. The method of claim 1, wherein the dataset of clinical outcome prediction scores of the plurality of individuals are arranged as an n-dimensional Euclidean space, wherein each of the n dimensions denotes an axis according to a respective clinical outcome prediction indicative of a certain pathology of each certain physiological system, wherein each individual is represented as a point in the n-dimensional Euclidean space according to respective prediction scores of each axis, wherein the computing the cluster is performed by mapping the set of clinical outcome prediction scores for the target individual to a point in the n-dimensional Euclidean space and computing the nearest neighbors based on closest Euclidean distance between the point denoting each individual and the point denoting the target individual.

3. The method of claim 2, wherein the requirement of the statistical distance defines a predefined number of nearest neighbors according to Euclidean distances between a point in the n-dimensional space denoting the mapped set of clinical outcome prediction scores for the target individuals and points in the n-dimensional space noting respective locations of each of the members of the cluster.

4. (canceled)

5. The method of claim 1, wherein the clinical outcome prediction scores of the target individual are indicative of the probability of the target individual developing the corresponding predicted certain pathology of the certain physiological system within a predefined future time interval.

6. The method of claim 1, further comprising excluding from the plurality of individuals, individuals having indications of medical test results separated from a defined clinical outcome stored in a medical database by greater than a predefined interval of time.

7. (canceled)

8. The method of claim 1, further comprising computing the trained classifier by: extracting from a medical database, for a subset of the plurality of individuals, at least one set of indications of medical test results and at least one indication of a diagnosis of a clinical outcome, wherein individuals without the diagnosis of the clinical outcome are labeled as negative individuals denoting lack of association with the clinical outcome and individuals associated with the diagnosis are labeled as positive individuals denoting an association with the clinical outcome;
creating a training dataset by sampling a defined ratio of individuals labeled as positive individuals and individuals labeled as negative individuals; and
computing the trained classifier according to the training dataset.

9. The method of claim 8, further comprising for individuals labeled as positive individuals filtering out members of the set of indications of medical test results with dates that are outside of a defined time interval relative to the date of the diagnosis of clinical outcome, and for individuals labeled as negative individuals filtering out members of the set of indications of medical test results with dates that are within the defined time interval relative to the date of computation of the trained classifier.

10. The method of claim 8, further comprising:
accessing from a medical database, for each of the subset of the plurality of individuals, at least one of: at least one demographic parameter and at least one prescribed medication; and
including the at least one of: the at least one demographic parameter and the at least one prescribed medication in the training dataset.

11. The method of claim 8, further comprising computing the dataset storing the set of clinical outcome prediction scores, by applying the trained classifier to compute the clinical outcome prediction scores for each member of a validation dataset including individuals of the medical database excluded from the training dataset.

12. The method of claim 1, further comprising:
designating a calibration set of a plurality of individuals associated with medical test results stored in a medical database;
applying the trained classifier to the calibration set to compute a first set of clinical outcome prediction scores;
applying a demographic classifier to demographic data of the calibration set to compute a second set of clinical outcome prediction scores, wherein the demographic classifier computes clinical outcome prediction scores according to demographic data of a certain individual;
sorting the first and second set of computed clinical outcome prediction scores computed for the calibration set;
dividing the sorted clinical outcome prediction scores into bins;
computing the prevalence of each clinical outcome prediction indicative of a certain pathology of each certain physiological system in the calibration set; and
calibrating each bin according to the computed prevalence of clinical prediction outcomes of the respective bins.

13. The method of claim 1, further comprising:
computing a Fisher statistical significance of prevalence of clinical outcome predictions indicative of a certain pathology of each certain physiological system of the cluster relative to clinical outcome predictions of a demographic classifier applied to the demographic data of the target individual,
wherein the demographic classifier computes clinical outcome predictions according to demographic data of a certain individual; and
excluding clinical outcome predictions with Fisher p-values above a threshold, wherein the remaining clinical outcome predictions denote an elevated risk of the target individual developing the remaining clinical outcome predictions.

14. The method of claim 1, further comprising:
computing a ratio of the prevalence of clinical outcome predictions indicative of a certain pathology of each certain physiological system of the cluster relative to clinical outcome prediction scores computed by applying a demographic classifier to the demographic data of the target individual,
wherein the demographic classifier computes clinical outcome prediction scores according to demographic data of a certain individual; and
excluding clinical outcome prediction scores having a computed ratio below a predefined value, wherein the remaining clinical outcome prediction scores denote an elevated risk of the target individual developing the remaining clinical outcome predictions.

15. The method of claim 1, further comprising identifying clinical outcome predictions indicative of a certain pathology of each certain physiological system with a prediction score value above a requirement, wherein the identified clinical outcome prediction scores denote an absolute risk of the target individual developing the respective clinical outcome prediction.

16. The method of claim 1, wherein the certain pathology of each certain physiological system include one or more members selected from the group consisting of: death, neoplasm, ischemic heart disease, type two diabetes, liver disease, chronic renal failure, chronic obstructive pulmonary disease, acquired hypothyroidism, epilepsy, migraine, chronic fatigue syndrome, trigeminal neuralgia, hypertensive disease, nervous system disease, acute sinusitis, bell facial palsy, carpal tunnel syndrome, retinal detachment, diabetic retinopathy, degeneration of macula, glaucoma, vertiginous syndromes, hyperthyroidism, acute renal failure, heart valve disorders, haematuria, psoriasis, systemic lupus erythematosus, polymyalgia rheumatic, pulmonary embolism, cataract, cardiac dysrhythmias, and osteoporosis.

17. The method of claim 1, wherein computing the aggregation of at least one medical parameter comprises computing at least one of:
a prevalence for at least one clinical outcome prediction indicative of a certain pathology of each certain physiological system for the cluster of clinically similar individuals; and
a difference between a prevalence of at least one clinical outcome prediction computed for the cluster in comparison to the prevalence of the at least one clinical outcome prediction computed for a general population that is demographically correlated with the target individual,
wherein the method further comprises generating an indication of the at least one clinical outcome prediction when the prevalence of the at least one clinical outcome prediction is statistically significant between the cluster and the general population.

18. (canceled)

19. The method of claim 1, wherein computing the aggregation comprises computing at least one of:
an average age of members of the cluster of clinically similar individuals, wherein the average age of members of the cluster of clinically similar individuals denotes a biological age of the target individual; and
an average age of members of the cluster of clinically similar individuals that are correlated according to a requirement with at least one clinical prediction indicative of a certain pathology of each certain physiological system computed for the target individual, wherein the computed average age denotes a biological age of at least one of an organ and a physiological system of the target individual associated with each respective clinical prediction.

20. (canceled)

21. The method of claim 1, wherein computing the aggregation of the at least one medical parameter comprises for at least one of an indication of at least one of a medical treatment, a prescribed medication, and a specialist consultation of the target individual, computing at least one of:
a difference between an incidence of at least one of the medical treatment, the prescribed medication, and the specialist consultation computed for the cluster in comparison to an incidence of at least one of the medical treatment, the prescribed medication, and the specialist consultation computed for a general population that is demographically correlated with the target individual,
the effect of the contribution of at least one of the medical treatment, the prescribed medication, and the specialist consultation in a difference between a prevalence of at least one clinical outcome prediction computed for the cluster in comparison to the prevalence of the at least one clinical outcome prediction computed for a general population that is demographically correlated with the target individual;
wherein the method further comprises generating an indication of the at least one of the medical treatment, the prescribed medication, and the specialist consultation when the incidence of the at least one of the medical treatment, the prescribed medication, the specialist consultation is statistically significant between the cluster and the general population, and the specialist consultation when the effect of the contribution on the prevalence of the at least one clinical outcome prediction is statistically significant between the cluster and the general population.

22. (canceled)

23. The method of claim 1, wherein computing the aggregation of the at least one medical parameter comprises for at least one of a value and a trend of values of an analyte included in the indication of laboratory test of the medical test results of the target individual,
computing the effect of the contribution of at least one of the value and the trend of values of the analyte in a difference between a prevalence of at least one clinical outcome prediction computed for member of the cluster having similar at least one value and trend of the analyte, in comparison to the prevalence of the at least one clinical outcome prediction computed for a general population that is demographically correlated with the target individual,
and generating an indication of the at least one of the value and the trend of the analyte and the at least one clinical outcome prediction when the effect of the contribution on the prevalence of the at least one clinical outcome prediction is statistically significant between the cluster and the general population.

24. The method according to claim 1, wherein the clinical outcome prediction scores are computed for the target individuals and the plurality of individuals according to the indication of medical test results and indications of determinant of health data including one or more of: demographic data, genetic data, nutrition, and environmental exposure.

25. (canceled)

26. A system for providing a client terminal with an aggregation of at least one medical parameter of members of a cluster of individuals clinically similar to a target individual, comprising:
a non-transitory memory having stored thereon a code for execution by at least one hardware processor of a computing system in communication with a dataset storing a set of computed clinical outcome prediction scores for each of a plurality of individuals, the code comprising:
code for receiving an indication of medical test results of the target individual;
code for applying a trained classifier to the indication of medical test results to compute, for the target individual, a set of clinical outcome prediction scores wherein each respective score is indicative of a prediction of a certain pathology of a plurality of pathologies of a certain physiological system of a plurality of physiological systems of the target individual, computing for the set of clinical outcome prediction scores of the target individual, a cluster of sets of computed clinical output predictions stored by the dataset, wherein the cluster of sets of computed clinical outcome predictions is computed according to a requirement of a statistical distance of sets of computed clinical outcome prediction scores stored by the dataset relative to the set of clinical outcome prediction scores of the target individual, wherein each set of clinical outcome prediction scores is indicative of a predicted co-morbidity of the plurality of pathologies of the plurality of physiological systems, wherein the individuals defined by the computed cluster denote individuals clinically similar to the target individual in terms of predicted co-morbidity of the plurality of pathologies of the plurality of physiological systems, and computing an aggregation of at least one medical parameter of the cluster; and code for outputting the aggregation for presentation by the client terminal.

27. (canceled)
